(19) United States
(12) Patent Application Publication
(10) Pub. No.: US 2014/0220625 A1
(43) Pub. Date: Aug. 7, 2014

(54) UBIQUITIN CHAIN ASSEMBLY

(71) Applicant: Medical Research Council, Swindon (GB)

(72) Inventors: Manuela K. Hospenthal, Cambridge (GB); David Komander, Cambridge (GB)

(73) Assignee: Medical Research Council, Swindon (GB)

(21) Appl. No.: 14/346,169

(22) PCT Filed: Sep. 24, 2012
(86) PCT No.: PCT/EP2012/003988

§ 371 Date: Mar. 20, 2014

(30) Foreign Application Priority Data
Sep. 23, 2011 (GB) 1116528.9

(51) Int. Cl. C12P 21/02

(52) U.S. Cl. CPC C12P 21/02
USPC 435/68.1; 435/212

(57) ABSTRACT

There is provided a method for producing free polyubiquitin chains linked through a single desired lysine residue, comprising the steps of: (a) selecting an E3 ubiquitin ligase enzyme which is homologous to mammalian HECT E3 ligases and possesses the desired lysine residue specificity; (b) incubating the E3 enzyme with an E1 ubiquitin activating enzyme, an E2 ubiquitin conjugating enzyme and monomeric ubiquitin; and (c) if undesired linkages are present, removing the undesired linkages by exposure to a DUB enzyme having the appropriate specificity.

The present invention relates to assembly of polyubiquitin chains having a determined linkage. In particular, the invention relates to the use of bacterial E3 ligases such as NleL to direct chain assembly. Linkage specificity is controlled by preventing or antagonising formation of undesired linkage types.

### INTRODUCTION

Protein ubiquitination is a versatile posttranslational modification with roles in protein degradation, cell signalling, intracellular trafficking and the DNA damage response (Chen and Sun, 2009; Komander, 2009). Ubiquitin polymers are linked through one of seven internal lysine (K) residues or through the N-terminal amino group. Importantly, the type of ubiquitin linkage determines the functional outcome of the modification chains. Possible linkages include K6, K11, K27, K29, K33, K48 and K63 linkages, or linkage to the N-terminus of a proximal unit (Komander, 2009). The best-studied ubiquitin polymers, K48- and K63-linked chains, have degradative and non-degradative roles, respectively (Chen and Sun, 2009; Hershko and Ciechanover, 1998). Recent data has revealed an unexpected high abundance of so-called atypical ubiquitin chains; for example, K11 linkages have been found to be as abundant as K48-linkages in S. cerevisiae (Peng et al., 2003; Xu et al., 2009). In our International patent application PCT/GB2011/000704 we describe the synthesis of K11 linked polyubiquitin.

Very little is known about the remaining linkage types. K6-linked Ub chains represent an enigmatic chain type that is readily detected in yeast (Xu et al, 2009) and mammalian cells (Dammer et al, 2011). This chain type has been linked to DNA repair processes since the BRCA1/BARD1 Ub ligase complex was reported to assemble K6-linkages (Morris & Solomon, 2004; Nishikawa et al, 2004; Wu-Baer et al, 2003). The related Ring1B/Bmi1 polycomb E3 ligase complex assembles heterotypic Ub chains with branches at K6, K27 and K48 (Ben-Saadon et al, 2006). However the cellular roles of K6-linkages are currently unclear, and further insight into K6-linkages has been hindered by the lack of tools. We recently reported chemical synthesis of K6-linked Ub dimers (K6 diUb), and preliminary structural insights into this chain type suggested novel compact chain conformations (Virdee et al, 2010). However, enzymatic systems to assemble, bind or disassemble K6-linkages in vitro have not been reported.

Ubiquitin chains are assembled by an enzymatic cascade comprising E1 Ub activating, E2 Ub conjugating, and E3 Ub ligating enzymes (Dye & Schulman, 2007). Two mechanistic classes of E3 ligases transfer Ub either directly to a substrate (RING and U-box E3s, (Deshaies & Joazeiro, 2009)) or first form a stable thioester intermediate with Ub (HECT (Rotin & Kumar, 2009) and members of RING-in-between-RING family (Wenzel et al, 2011)) before substrate modification. Once a Ub chain is formed, it is recognized by Ub receptors that contain Ub binding domains (UBDs) (Dikic et al, 2009) and mediate downstream effects such as shuttling the protein to the proteasome. Ubiquitination is reversible and deubiquitinases (DUBs) hydrolyse Ub chains (Komander et al, 2009a; Reyes-Turcu et al, 2009). Importantly, assembly, recognition and hydrolysis of Ub chains can be highly linkage specific, suggesting non-redundant and/or regulated functions of the individual chain types (Komander, 2009).

HECT E3 ligases are known in the art, and represent a major family of ubiquitin ligases. They include the HERC ligases, KIAA10, Nedd4, E6AP, HECTD2, KIAA0614, TRIP12, G2E3, EDD, HACE1, HECTD1, UBE3B, UBE3C, KIAA0317, HUWE1 and HECTD3.

Pathogenic bacteria are known to deliver effector proteins into eukaryotic host cells to function as E3 ligases (Collins and Brown (2010) Trends Cell Biol. 20, 205-213; Hicks and. Gala'n, J. E. (2010) Curr. Opin. Microbiol. 13, 41-46; Angot, et al., (2007) PLoS Pathog. 3, e3; Munro, et al. (2007) Curr. Opin. Microbiol. 10, 39-46; Spallek, et al., (2009) Cell Microbiol. 11, 1425-1434). For example, SopA from Salmonella is an E3 ligase structurally similar to eukaryotic HECT E3s (Zhang, et al., (2006) Mol. Microbiol. 62, 786-793; Diao, et al. (2008) Nat. Struct. Mol. Biol. 15, 65-70).

Another bacterial Ub E3 ligase related to HECT family enzymes has been shown to assemble K6- and K48-chains in vitro (Lin et al, 2010). The protein NleL from Enterohemorrhagic Escherichia coli (EHEC) O157:H7 is a Type III effector that is injected into host cells by a type III secretion system (Piscatelli et al, 2011).

### SUMMARY OF THE INVENTION

We have developed a method for generating polyubiquitin chains that employs Ub ligases to produce unattached ubiquitin polymers.

Accordingly, there is provided a method for producing free polyubiquitin chains linked through a single desired lysine residue, comprising the steps of: (a) selecting an E3 ubiquitin ligase enzyme which is homologous to mammalian HECT E3 ligases and possesses the desired lysine residue specificity; (b) incubating the E3 enzyme with an E1 ubiquitin activating enzyme, an E2 ubiquitin conjugating enzyme and monomeric ubiquitin; and (c) if undesired linkages are present, removing the undesired linkages by exposure to a DUB enzyme having the appropriate specificity.

E3 ligases with HECT homology are known, and described for example from Huibregtse et al., Proc Natl Acad Sci USA 92: 2563-2567; and Schwarz et al., (1998) J Biol Chem 273: 12148-12154. For example, KIAA10 (Wang et al., 2006) catalyses the formation of K29 and K48-linked polyubiquitin chains.

Bacterial enzymes which polymerise mammalian ubiquitin are known. Enzymes which have homology to mammalian HECT E3 ligases include NleL, and SopA. In one embodiment, the bacterial enzyme is NleL.

NleL catalyses the polymerisation of monomeric ubiquitin into freestanding polyubiquitin chains. The preferred linkages are K6 and K48. In order to select a single desired linkage from K48 and K6, a DUB can be used to disassemble the other, undesired linkage; for example, if only K6 linkages are required, OTUB1 can be included to disassemble K48 linkages. If only K48 linkages are desired, OTUD3 can be included, which disassembles K6 (and K11) linkages.

In one embodiment, the DUB enzyme is used after the polymerisation reaction; for example, the NleL reaction is allowed to proceed, creating K48 and K6 linkages, and the K48 linkages are subsequently removed. In an alternative embodiment, the K6 linkages can be removed.

A table of DUB enzymes and respective linkage specificities is set forth below.

In one embodiment, the monomeric ubiquitin can be modified to prevent undesired linkage formation. This has the advantage of preventing the formation of linkages ab initio, and therefore removing the need to disassemble these linkages using DUB. If the use of a modified ubiquitin is consistent with the end-use envisaged for the polyubiquitin chain created, the use of modified ubiquitin is indicated.

Ubiquitin modifications include, for example, modification of the Lys residue(s) susceptible to linkage formation by the E3 used, but at which it is not desired to form a linkage, by conversion to another amino acid. In one embodiment, a K-R mutation is indicated. For example, K48R ubiquitin is not susceptible to K48 linkage formation by NleL.

The E1 and E2 enzymes included in the reaction can be any suitable E1 and E2 enzymes. E1 enzymes are available commercially, for instance from Enzo Lifesciences. A list of E1 enzymes is set forth in Table 1 of WO2004020674. A list of known E2 enzymes, identified by human gene names together with yeast homologue names, appears in Table S1 in the supplementary information supplied with Ye & Rape, 2009. In one embodiment, the E2 enzyme is Ube2L3/UbcH7.

In another aspect, there is provided kit for preparing polyubiquitin chains having a desired linkage specificity, comprising one or more E3 ubiquitin ligase enzymes which is homologous to mammalian HECT E3 ligases and possesses the desired lysine residue specificity, an E1 ubiquitin activating enzyme, an E2 ubiquitin conjugating enzyme and one or more DUB enzyme.

In one embodiment, the E3 ubiquitin ligase enzyme is NleL. For example, the DUB enzyme is OTUB1. In one embodiment, the E2 enzyme is Ube2L3/UbcH7.

The kit can also include one or more modified ubiquitin monomers, in which one or more lysine residues have been mutated to prevent linkage formation at said residue. For example, K48R mutants of ubiquitin cannot form K48 linkages.

### BRIEF DESCRIPTION OF THE FIGURES

FIG. 1: Ub chain assembly by NleL

(a) NleL was used in Ub chain assembly reactions with E1, UBE2L3/UbcH7, and Ub mutants as indicated. M, marker; wt, wild-type Ub; Ub-Lys only mutants, Ub mutants with Arg mutations in six out of seven Lys residues; Lys less, Ub harboring Arg mutations at all Lys residues. (b) A comparison of assembled unanchored Ub chains from wt Ub, Ub K6R and Ub K48R. Ub K6R and K48R lead to the assembly of Lys48 and Lys6 chains respectively. A Ub K6R/K48R double mutant is unable to assemble similar unanchored Ub products. Asterisks (*) label pentaUb species.

FIG. 2: Ubiquitin chain analysis

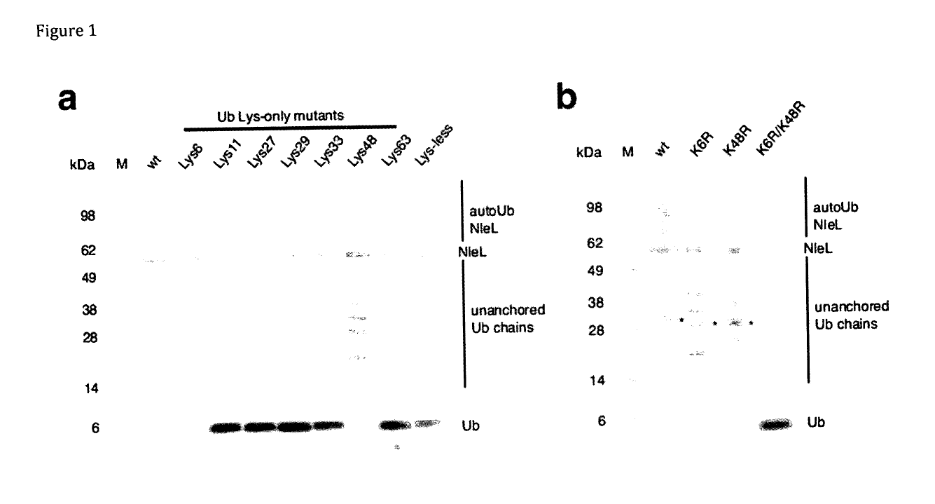

A) Schematic illustrating potential tetramer complexity in NleL assembled wt Ub products, and the concept of Ub chain sequencing. Ub (yellow square) can be linked either via Lys6 (blue line; horizontal) or Lys48 (red line; vertical), leading to 14 distinct species (boxed). An orange dot indicates the free C-terminus of Ub (proximal moiety). Right, linkage-specific DUBs can be used as ‘Ub chain restriction enzymes’ to reveal building blocks within heterotypic Ub chains. B) Specificity profile of OTUB1 and OTUD3 against diUb of all Ub linkages. A time course analysis is shown where enzyme was incubated with specified diUb for a given time, then resolved by SDS-PAGE and silver stained. C) Purified mixture of heterogeneous penta and hexaUb assembled by NleL from wt Ub (lane 1) is cleaved with OTUB1, OTUD3, vOTU or combinations thereof. A silver stained gel is shown. (D) Time-course analysis of NleL assembly reaction (as in FIG. 1a) with Ub K6R, Ub K48R and a combination of Ub ΔG76 and Ub K6R/K48R in a 1:2 molar ratio. (E) Schematic depicting the assembly of a branched triUb harboring one Lys6- and one Lys48-linkage. (F) NleL mediated assembly of Ub chains from Ub K6 only, Ub K48only and combination of Ub ΔG76 and Ub K6R/K48R in a 1:2 molar ratio. Resulting species are labeled. (G) Hydrolysis of branched triUb with OTUB1, OTUD3 and vOTU. A silver-stained SDSPAGE gel is shown.

FIG. 3: Large-scale assembly and purification of K6-linked polyUb.

(a) Time-course analysis of the assembly reaction of Lys6 polyUb by NleL using 25 mg Ub K48R as input material. (b) Chromatogram of purification of Lys6 polyUb species by size-exclusion chromatography (SEC). (c) Selected fractions corresponding to individual peaks from SEC purification in b are resolved by SDS-PAGE. (d) Comparison of the assembly reactions performed by NleL using wt Ub (lane 1), Ub K48R (lane 2) and Ub K48R subsequently treated using OTUB1 (lane 3). (e) Lys6-, Lys11-, Lys48-, Lys63- and Met1 tetraUb have distinct electrophoretic mobility on 4-12% SDS-PAGE gradient gels.

### DETAILED DESCRIPTION OF THE INVENTION

Unless defined otherwise, all technical and scientific terms used herein have the same meaning as commonly understood by those of ordinary skill in the art, such as in the arts of peptide chemistry, cell culture, nucleic acid chemistry and biochemistry. Standard techniques are used for molecular biology, genetic and biochemical methods (see Sambrook et al., Molecular Cloning: A Laboratory Manual, 3rd ed., 2001, Cold Spring Harbor Laboratory Press, Cold Spring Harbor, N.Y.; Ausubel et al., Short Protocols in Molecular Biology (1999) 4^{th} ed., John Wiley & Sons, Inc.). All publications cited herein are incorporated herein by reference in their entirety for the purpose of describing and disclosing the methodologies, reagents, and tools reported in the publications that might be used in connection with the invention.

A free polyubiquitin chain is a chain of at least two ubiquitin monomers covalently linked together and not attached to any other polypeptide or protein.

Linkage, in the present context, refers to the linkage formed between ubiquitin monomers in a polyubiquitin chain. In general, linkage is formed between a defined Lys residue in one ubiquitin molecule or through the N-terminal amino group. A single desired lysine residue refers to one of K6, K11, K27, K29, K48 and K63.

HECT E3 ligases are E3 ligases characterised by the HECT (homologous to E6AP C-terminus) domain, a C-terminal region of approximately 350 amino acids in length with significant similarity to the C-terminus of E6AP. See Scheffner & Staub, 2007, BMC Biochemistry 8(Suppl. 1): S6; Huibregtse et al., Proc Natl Acad Sci USA 92: 2563-2567; and Schwarz et al., (1998) J Biol Chem 273: 12148-12154.

E2 enzymes, as referred to herein, are variously known as ubiquitin carrier proteins, ubiquitin conjugating enzymes or Ubcs. In many instances, E2 enzymes are thought to determine linkage specificity in polyubiquitin. 38 E2 enzymes have been identified in humans, as described in Ye and Rape, 2009.

E1 enzymes, as referred to herein, are ubiquitin activating enzymes and are understood to catalyse the first step in a ubiquitination reaction. E1 enzymes are available commercially, for example as set for the herein.

DUB enzymes are deubiquitinase enzymes, and reviewed in Komander, 2009. We describe herein linkage-specific DUBs which are useful in disassembling particular ubiquitin linkages. For example, OUT domain DUBs are provided with specificities for any desired linkage.

Enzymes

Enzymes for use in conjunction with the present invention are known in the art and can be produced by conventional means. A number of necessary enzymes are available commercially, including E1 and E2 enzymes. Sources of E1 and E2 enzymes are described herein, and the documents referred to are incorporated herein by reference.

E3 ligases are also known in the art, and can be obtained by cloning of known sequences into suitable cloning and expression vectors. Sequences can be derived from the references set forth herein, the contents of which are incorporated by reference. Sequences can also be found in publically available databases, such as Genbank.

DUB enzymes are known in the art, and can be obtained and produced as for E1, E2 and E3 enzymes. Linkage-specific DUBs have been characterised herein for the first time; the following table describes the linkage specificity of DUB enzymes useful in the present invention.

The linkages cleaved by various enzymes are set forth in Table 1.

TABLE 1
The DUBs are identified as full length (FL) or individual
domains. 3 = preferred chain linkage cleavage; 2 =
some cleavage; 1 = negligible cleavage; 0 = no cleavage.

| DUB | K6 | K11 | K27 | K29 | K33 | K48 | K63 | M1 |
|---|---|---|---|---|---|---|---|---|
| OTUD1 | 0 | 0 | 0 | 0 | 0 | 0 | 3 | 0 |
| FL | | | | | | | | |
| OTUD1 | 0 | 0 | 0 | 0 | 0 | 0 | 3 | 0 |
| OTU + | | | | | | | | |
| UIM | | | | | | | | |
| OTUD1 | 0 | 2 | 3 | 3 | 3 | 3 | 2 | 0 |
| OTU | | | | | | | | |
| OTUD1 | 3 | 3 | 1 | 1 | 1 | 3 | 3 | 0 |
| tOTU + | | | | | | | | |
| UIM | | | | | | | | |
| OTUD2 | 1 | 2 | 3 | 3 | 3 | 2 | 0 | 0 |
| FL | | | | | | | | |
| OTUD2 | 1 | 2 | 3 | 3 | 3 | 2 | 0 | 0 |
| OTU + | | | | | | | | |
| ZnF | | | | | | | | |
| OTUD2 | 0 | 3 | 1 | 1 | 1 | 2 | 0 | 0 |
| OTU | | | | | | | | |
| OTUD3 | 3 | 3 | 1 | 1 | 1 | 1 | 1 | 0 |
| OTU + | | | | | | | | |
| UBA | | | | | | | | |
| OTUD3 | 3 | 3 | 1 | 1 | 1 | 1 | 1 | 0 |
| OTU | | | | | | | | |
| OTUD4 | 1 | 1 | 1 | 0 | 0 | 3 | 1 | 0 |
| OTU | | | | | | | | |
| OTUD5 | 0 | 0 | 0 | 0 | 0 | 0 | 0 | 0 |
| OTU | | | | | | | | |
| OTUD5 | 2 | 2 | 1 | 1 | 1 | 3 | 3 | 0 |
| pOTU | | | | | | | | |
| OTUD6A | 0 | 2 | 3 | 3 | 3 | 2 | 1 | 0 |
| FL | | | | | | | | |
| OTUD6B | 0 | 0 | 0 | 0 | 0 | 0 | 0 | 0 |
| FL | | | | | | | | |
| OTUB1 | 0 | 0 | 0 | 0 | 0 | 3 | 0 | 0 |
| FL | | | | | | | | |
| OTUB2 | 0 | 2 | 0 | 0 | 0 | 2 | 3 | 0 |
| FL | | | | | | | | |
| A20 OTU | 0 | 2 | 0 | 0 | 0 | 3 | 0 | 0 |
| Cezanne | 0 | 3 | 0 | 0 | 0 | 0 | 0 | 0 |
| OTU | | | | | | | | |
| Cezanne | 0 | 3 | 0 | 0 | 0 | 0 | 0 | 0 |
| 2 OTU | | | | | | | | |
| TRABID | 0 | 0 | 0 | 3 | 3 | 0 | 2 | 0 |
| OTU + | | | | | | | | |
| AnkUBD | | | | | | | | |
| VCPIP | 0 | 3 | 0 | 0 | 0 | 3 | 0 | 0 |
| OTU | | | | | | | | |
| OTULIN | 0 | 0 | 0 | 0 | 0 | 0 | 0 | 3 |

Other DUBs are known which have a lower level of specificity. These include USP21 (Ye et al EMBO Rep, 2011); viral OTU domains (Akutsu et al, PNAS 2011).

Recombinant Expression of Enzymes

Expression of nucleic acids encoding DUBs, E1, E2 or E3 can be carried out in any suitable expression system. Expressions systems are known in the art and may be obtained commercially or according to instructions provided in laboratory manuals.

A wide variety of expression systems are available for the production of chimeric polypeptides. For example, expression systems of both prokaryotic and eukaryotic origin may be used for the production of enzymes.

Nucleic acid vectors are commonly used for protein expression. The term “vector” refers to a nucleic acid molecule that may be used to transport a second nucleic acid molecule into a cell, and/or express it therein. In one embodiment, the vector allows for replication of DNA sequences inserted into the vector. The vector may comprise a promoter to enhance expression of the nucleic acid molecule in at least some host cells. Vectors may replicate autonomously (extrachromosomal) or may be integrated into a host cell chromosome. In one embodiment, the vector comprises an expression vector capable of producing a fusion protein derived from at least part of a nucleic acid sequence inserted into the vector.

A cloning vector can be a nucleic acid molecule, such as a plasmid, cosmid, or bacteriophage, that has the capability of replicating autonomously in a host cell. Cloning vectors typically contain one or a small number of restriction endonuclease recognition sites that allow insertion of a nucleic acid molecule in a determinable fashion without loss of an essential biological function of the vector, as well as nucleotide sequences encoding a marker gene that is suitable for use in the identification and selection of cells transformed with the cloning vector. Marker genes typically include genes that provide tetracycline resistance or ampicillin resistance.

An expression vector typically comprises a transcription promoter, a gene, and a transcription terminator. Expression vectors may be autonomously replicating, or integrated into the host genome. Gene expression is usually placed under the control of a promoter, and such a gene is said to be operably linked to the promoter. Similarly, a regulatory element and a core promoter are operably linked if the regulatory element modulates the activity of the core promoter. The nucleic acid encoding the chimeric enzyme according to the invention is typically expressed under the control of a promoter in an expression vector.

To express a gene, a nucleic acid molecule encoding the protein must be operably linked to regulatory sequences that control transcriptional expression and then, introduced into a host cell. In addition to transcriptional regulatory sequences, such as promoters and enhancers, expression vectors can include transcriptional and translational regulatory sequences. The sequences used will be appropriate to the host, which may be prokaryotic or eukaryotic. The transcriptional and translational regulatory signals suitable for a mammalian host may be derived from viral sources, such as adenovirus, bovine papilloma virus, simian virus, or the like, in which the regulatory signals are associated with a particular gene that has a high level of expression. Suitable transcriptional and translational regulatory sequences also can be obtained from mammalian genes, such as actin, collagen, myosin, and metallothionein genes. Prokaryotic regulatory sequences may similarly be derived from viral genes, and are known in the art.

The inclusion of an affinity tag is useful for the identification or selection of cells expressing the fusion protein. Examples of affinity tags include polyHistidine tags (which have an affinity for nickel-chelating resin), c-myc tags, which are detected with anti-myc antibodies, calmodulin binding protein (isolated with calmodulin affinity chromatography), substance P, the RYIRS tag (which binds with anti-RYIRS antibodies), a hemagglutinin A epitope tag, which is detected with an antibody, the Glu-Glu tag, and the FLAG tag (which binds with anti-FLAG antibodies). Nucleic acid molecules encoding such peptide tags are available, for example, from Sigma-Aldrich Corporation (St. Louis, Mo., USA).

The gram-negative bacterium E. coli is widely used as a host for heterologous gene expression. Although large amounts of heterologous protein can accumulate inside the cell, this expression system is effective in the context of the present invention. Suitable strains of E. coli include BL21(DE3), BL21(DE3)pLysS, BL21(DE3)pLysE, DH1, DH4I, DH5, DH5I, DH5IF′, DH5IMCR, DH10B, DH10B/p3, DH11S, C600, HB101, JM101, JM105, JM109, JM110, K38, RR1, Y1088, Y1089, CSH18, ER1451, and ER1647.

Bacteria from the genus Bacillus are also suitable as heterologous hosts, and have capability to secrete proteins into the culture medium. Other bacteria suitable as hosts are those from the genera Streptomyces and Pseudomonas. Suitable strains of Bacillus subtilus include BR151, YB886, MI119, MI120, and B170 (see, for example, Hardy, “Bacillus Cloning Methods,” in DNA Cloning: A Practical Approach, Glover (ed.) (IRL Press 1985)). Standard techniques for propagating vectors in prokaryotic hosts are well-known to those of skill in the art (see, for example, Ausubel 1995; Wu et al., Methods in Gene Biotechnology (CRC Press, Inc. 1997)).

Eukaryotic hosts such as yeasts or other fungi may be used. In general, yeast cells are preferred over fungal cells because they are easier to manipulate. However, some proteins are either poorly secreted from the yeast cell, or in some cases are not processed properly (e.g. hyperglycosylation in yeast). In these instances, a different fungal host organism should be selected.

The use of suitable eukaryotic host cells—such as yeast, fungal and plant host cells—may provide for post-translational modifications (e.g. myristoylation, glycosylation, truncation, lapidation and tyrosine, serine or threonine phosphorylation) as may be needed to confer optimal biological activity on recombinant expression products.

In some embodiments, the fusion proteins may be expressed as GST fusions. For example, the pGEX and pOPIN vector systems can employ a GST fusion. Use of GST as a fusion partner provides an inducible expressions system which facilitates the production of proteins in the E. coli system. Proteins expressed using this system can be isolated using a glutathione capture resin.

For example, recombinant GST fusion constructs are expressed in Rosetta 2 (DE3) pLacI cells (Novagen). 1 L cultures of cells are induced at OD₆₀₀ of 0.6 with 250 μM IPTG and proteins are expressed at 20° C. overnight. Cells are harvested and flash-frozen. 30 ml lysis buffer containing 270 mM sucrose, 50 mM Tris (pH 8.0), 50 mM NaF, 1 protease inhibitor cocktail tablet (Roche) (0.1% v/v β-mercaptoethanol, 1 mg/ml lysozyme and 0.1 mg/ml DNase) are added per liter of culture. After sonication, cell lysates are cleared using a Sorvall SS-34 rotor (18,000 rpm, 30 min, 4° C.) and supernatants are incubated with Glutathione Sepharose 4B (GE Healthcare) for 1 h to immobilize soluble GST fusion proteins. Subsequently, the sepharose beads are washed with 500 ml high salt buffer [500 mM NaCl, 25 mM Tris (pH 8.5), 5 mM DTT] and 300 ml low salt buffer [150 mM NaCl, 25 mM Tris (pH 8.5), 5 mM DTT]. For site-specific cleavage of the GST tag, immobilized fusion proteins are incubated with 30 mM PreScission protease (GE Healthcare) overnight. Cleaved proteins are eluted with low salt buffer and flash-frozen in liquid nitrogen. All samples are >95% pure after purification.

Conservative Enzyme Mutation

The invention contemplates the use of natural enzymes that have been mutated. Conservative amino acid substitutions generally follow the following scheme:

| Side chain | Members |
|---|---|
| Hydrophobic | met, ala, val, leu, ile |
| Neutral hydrophilic | cys, ser, thr |
| Acidic | asp, glu |
| Basic | asn, gln, his, lys, arg |
| Residues that influence chain orientation | gly, pro |
| Aromatic | trp, tyr, phe |

In the above table, amino acids identified in the same row are considered to have similar side-chains and are can be substituted for each other with the least impact on protein structure and function.

Mutation can be at the nucleic acid level, that is changes may be effected to the nucleic acid encoding a Ubc domain without changing the structure of the enzyme itself, as a result of redundancy in the genetic code. Such changes can, for example, confer improved expression in heterologous host cells by employing preferred codon usage patterns.

Other mutations will change the amino acid sequence of the enzyme. As noted above, this can take the form of additions to or deletions from the N and C termini of the protein. Moreover, changes may be made within the sequence of the enzyme, for example through substitution, addition or deletion of one or more amino acids. Conservative amino acid substitutions are preferred, as set forth above. For example, 1, 2, 3, 4, 5, 6, 7, 8, 9, 10, 15, 20 or more amino acids are added, deleted and/or substituted by other amino acids.

In a preferred embodiment, the naturally occurring enzyme is used.

Production of Free Polyubiquitin Chains

The methods and kits of the invention produce high levels of free polyubiquitin. Assays for ubiquitination are known in the art; for instance, a description of such assays, and relevant background, is set forth, for example, in WO2009134897, US2006088901 and WO2004020674. Ubiquitination assays kits are available commercially, for instance from Cisbio, Bedford, Mass., USA; Invitrogen, Carlsbad, Calif., USA; and Enzo Lifesciences, Plymouth Meeting, Pa., USA.

In general, the production of free ubiquitin requires the incubation of E1 enzyme, E2 enzyme and an E3 ligase as described herein, together with monomeric ubiquitin in the presence of ATP in a buffer solution.

In one embodiment, ubiquitin may be labelled, to facilitate its subsequent detection or isolation. In another embodiment, the ubiquitin may be modified to prevent undesired linkage formation.

In one embodiment, small-scale analytical ubiquitination assays can be carried out in 30 μL reactions by incubating 8 μg Ub or Ub Lys-only mutants (46 mM), 125 nM Ub activating enzyme (E1), 2.24 μM UBE2L3/UbcH7 Ub conjugating enzyme (E2), 1.5 μM NleL Ub ligase (E3), 10 mM ATP, 40 mM Tris (pH 7.5), 10 mM MgCl2, 0.6 mM DTT, at 37° C. for 30 min. The reaction can be stopped by the addition of 10 μL 4× LDS sample buffer (Invitrogen) and samples analyzed by SDS-PAGE, using 4-12% NuPAGE gradient gels with MES buffer (Invitrogen) and coomassie stained using Simply Blue Safe Stain (Expedeon protein solutions).

The reaction can alternatively be stopped by adding 5 μl 4M HCl to precipitate the enzymes, and the polyubiquitin chains purified by size exclusion chromatography.

Modified ubiquitin can be used, to prevent formation of undesired linkages.

Alternatively, or in addition, DUB enzymes can be added to the polyubiquitin preparation to disassemble undesired linkages.

Kits

Kits can be provided which facilitate the production of polyubiquitin chains having a single, desired linkage. Such kits advantageously comprise an E3 ubiquitin ligase enzyme which is homologous to mammalian HECT E3 ligases, an E1 ubiquitin activating enzyme, an E2 ubiquitin conjugating enzyme and a DUB enzyme. The DUB enzyme can be omitted, particularly if the kit is used with, or further comprises, one or more mutated ubiquitin monomers which are unable to form undesired linkage types.

In one embodiment, mutated ubiquitin monomers and DUB enzymes can be used in combination, to obtain a desired linkage.

Kits can also comprise packaging and instructions for use.

In one embodiment, a kit can further comprise one or more of ubiquitin and/or a mutant ubiquitin, ATP, Tris buffer (pH 7.5), MgCl₂ and DTT.

The components of the kit are advantageously provided in separate containers.

### EXAMPLES

Methods

Cloning and Mutagenesis

OTUD3 was amplified by PCR from and cloned using the In-Fusion® system (Clontech) into the pOPIN-K vector, which incorporates a Pre-Scission protease cleavable N-terminal glutathione-S-transferase (GST) tag (Berrow et al, 2007). GST-NleL 170-782 (pMCSG20) was a gift from David Yin-Wei Lin and Jue Chen (Lin et al, 2010). Ub mutants were generated by site-directed mutagenesis according to the QuikChange protocol but using KOD polymerase (Merck Chemicals).

Protein Production

Recombinant GST-NleL was expressed according to (Lin et al, 2010) in Rosetta 2 (DE3) pLacI cells (Novagen) harbouring a plasmid encoding rare codons. Cultures (4-6 L) were grown in LB media to OD₆₀₀ of 0.6-0.8 and induced using 0.1 mM IPTG at 16° C. for 24 h(NleL). All purifications were performed at 4° C. Cells were harvested and lysed by sonication in NleL Lysis buffer (50 mM Tris [pH 8.0], 500 mM NaCl, 10% (v/v) glycerol, 1 mM EDTA, 5 mM DTT, 0.1 mg/mL DNAse I, 1 mg/mL lysozyme, Complete Mini EDTA-free protease inhibitor cocktail (Roche)). Cell lysates were cleared by centrifugation (40000 g, 30 min, 4° C.) and the supernatants incubated with Glutathione Sepharose 4B (GE Healthcare) for 1-2 h to immobilise soluble GST-tagged proteins. The beads were subsequently washed with 1 L high salt buffer (50 mM Tris pH 8.0, 500 mM NaCl, 5 mM DTT), followed by 1 L buffer A (50 mM Tris pH 8.0, 150 mM NaCl, 5 mM DTT). The GST tag was cleaved by incubation of beads with recombinant Tobacco Etch Virus (rTEV) protease overnight. Cleaved proteins were eluted and further purified by anion exchange chromatography (RESOURCE Q, GE Healthcare), applying a linear NaCL gradient from 50-500 mM NaCl in 50 mM Tris pH 8.0, 5 mM DTT. NleL containing fractions were finally purified by size exclusion chromatography using a Superdex75 (GE Healthcare) in buffer A. The protein was concentrated using Amicon spin concentrators (10 kDa MW cut-off) and the purity was judged by SDS-PAGE analysis to be >95%.The same protocol was applied for OTUD3 with the differences that it was expressed at 20° C. overnight and lysed in OTUD3 lysis buffer (50 mM Tris pH 8.0, 200 mM NaCl, 5 mM DTT, 1 mg/mL lysozyme, 0.1 mg/mL DNAse I). His6-tagged OTUB1 was purified according to (Edelmann et al, 2009) and the CCHFV viral OTU domain according to (Akutsu et al, 2011). Ub mutants were expressed and purified as previously described (Pickart & Raasi, 2005).

In Vitro Deubiquitination Assays

DUBs were diluted to 0.1 mg/mL (5.5 μM) for OTUD3, 0.1 mg/mL (2.7 μM) for OTUB1, 0.2 μg/mL (9.5 nM) vOTU in Dilution buffer (25 mM Tris pH 7.5, 150 mM NaCl, 10 mM DTT). Subsequently, 20 μL of diluted DUB was mixed with 4 μg polyUb and 4 μL 10× DUB buffer (500 mM Tris pH 7.5, 500 mM NaCl, 50 mM DTT) in 40 μL reactions. At given timepoints, 5 μL of the reaction was stopped by mixing with 5 μL of 4× LSD sample buffer and analysed by SDS-PAGE. Protein was visualised using silver staining using the Silver Stain Plus Kit (Bio-Rad) following the manufacturer's protocol.

Large-Scale K6 Chain Assembly

Large-scale assembly of K6-linked polymers from K48R mutant Ub was carried out in 1 mL reactions from 25 mgUb K48R (2.91 mM), 0.1 μM E1, 0.56 μM UbE2L3, 11.1 μM NleL, 10 mM ATP, 40 mM Tris (pH 7.5), 10 mM MgCl₂, 0.6 mM DTT at 37° C. for 3-4 h. Subsequently, 5 μL of 4 M HCl was slowly added to the reaction, which reduces the pH to 4.0-5.0 and precipitates enzymes. The reaction was centrifuged (16000×g, 4° C.) and filtered through a 0.2 μm syringe filter before purification by size exclusion chromatography using a Superdex 75 16/60 column (GE Healthcare) in 50 mM Tris (pH 7.6). Peak fractions containing K6-linked diUb, triUb, tetraUb were pooled and concentrated using 3 kDa MW cut-off Amicon spin concentrators.

NMR Spectroscopy

NMR acquisition was carried out at 298 K on Bruker Avance III 600 MHz and Avance2+700 MHz spectrometers equipped with cryogenic triple resonance TCI probes. Data processing and analysis were carried out in Topspin (Bruker, Karlsruhe) and Sparky (Goddard & Kneller, University of California, San Francisco) respectively. ¹⁵N fast HSQC experiments ⁶² were used to measure weighted chemical shift perturbations which were defined as ((Δ¹H)²)^{0.5}+((Δ¹⁵N/5)²)^{0.5} [ppm] (Hajduk, P. J. et al (1997) J. Med. Chem. 40, 3144-3150). In the case of shifted or doubled peaks, standard triple resonance experiments (HNCACB, CBCA(CO)NH and HNCA) were used to unambiguously assign all ¹⁵N, ¹³C backbone resonances. For a semi-quantitative assessment of exchange broadening, the apparent peak attenuation was plotted as the peak height difference between perturbed and unperturbed species. No attempt was made to correct for potential changes in the overall correlation times.

### Example 1

NleL Assembles Heterotypic K6/K48 Chains In Vitro

It has been reported that NleL is able to assemble unattached K6- and K48-linked polyubiquitin in vitro (Lin et al, 2010). We confirmed that NleL assembles unattached chain with wild-type (wt) Ub, and the set of single-Lys Ub mutants showed NleL specificity for K6 and K48 (FIG. 1a). Ub mutation of either K6 or K48 to Arg still resulted in free Ub chains (of presumably the other type), while a double mutant K6/48R was unable to assemble free chains (FIG. 1a).

Electrophoretic mobility of Ub chains with three or more Ub molecules varies with linkage type, which can be used diagnostically (Komander et al, 2009b). NleL assembled diUb from wt Ub resulted in a double band indicating different electrophoretic mobility of K6 and K48 diUb. Interestingly, longer chains formed diffuse bands with wt Ub (FIG. 1b), suggesting heterotypic chains containing branches or alternating linkage types. Ub K6R or K48R mutation resulted in sharp well-defined bands of distinct electrophoretic mobility, revealing that a single chain type was formed (FIG. 1b).

### Example 2

Identification of Linkage Specific DUBs

Despite the observed K6-linkage assembly in vitro using the K48R mutant Ub, the amount and length of K6 vs. K48-linked Ub chains in NleL conjugates with Ub was unclear. It was considered possible that K6-linkages were an insignificant by-product of the K48 ligase NleL. However, the architecture of heterotypic Ub chains is difficult to assess by current technologies such as mass-spectroscopy (Dammer & Peng, 2010).

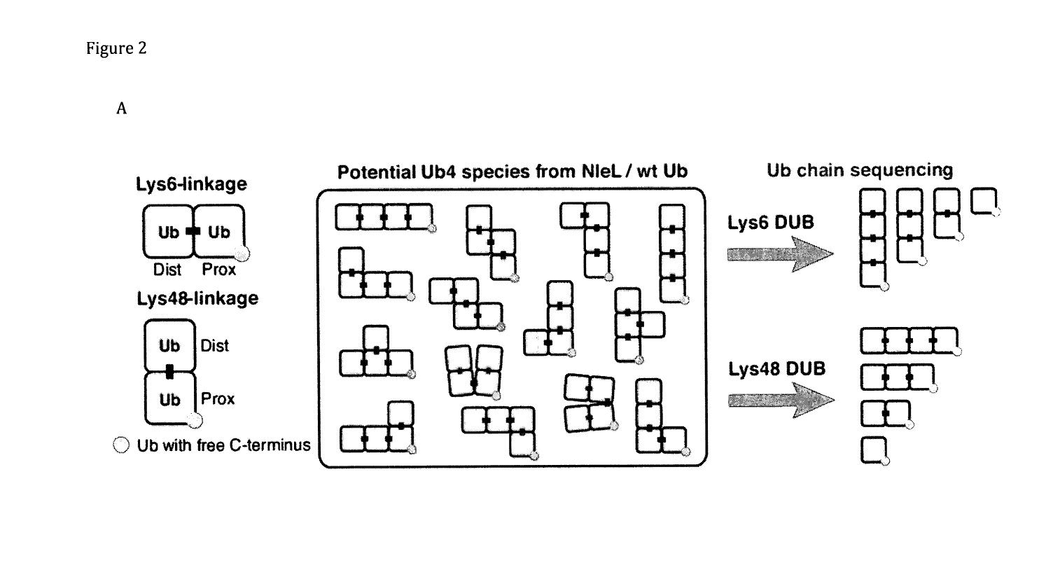

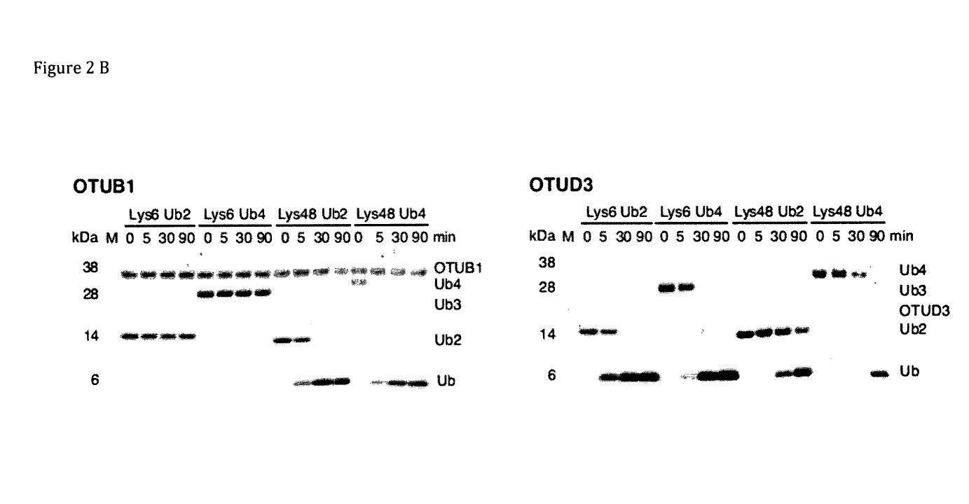

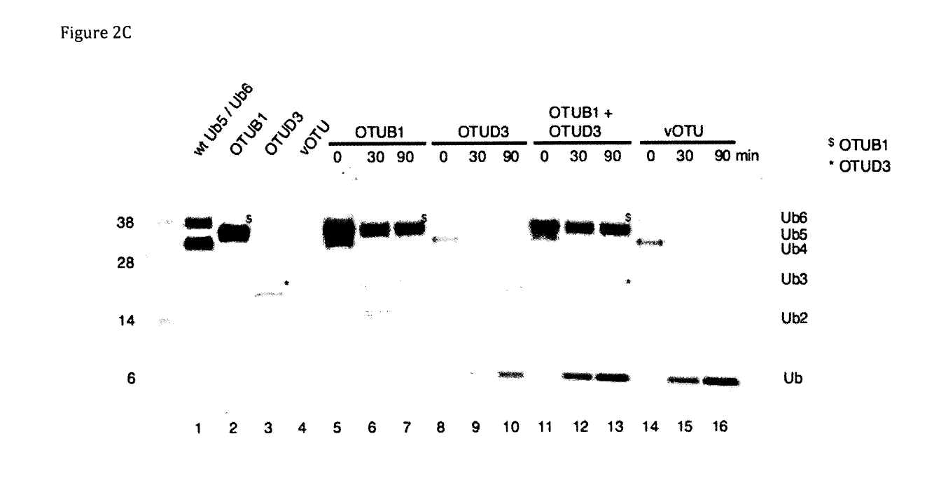

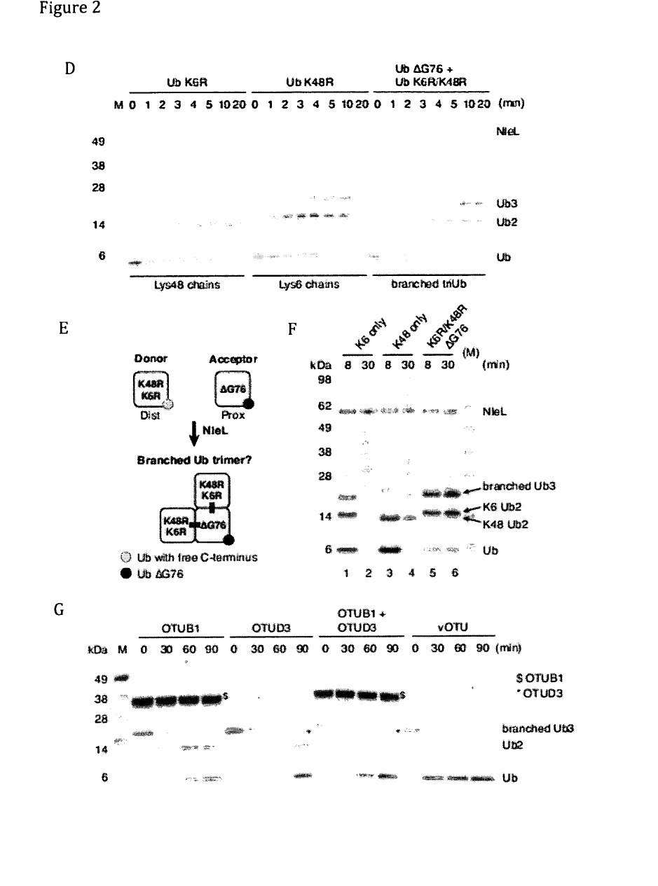

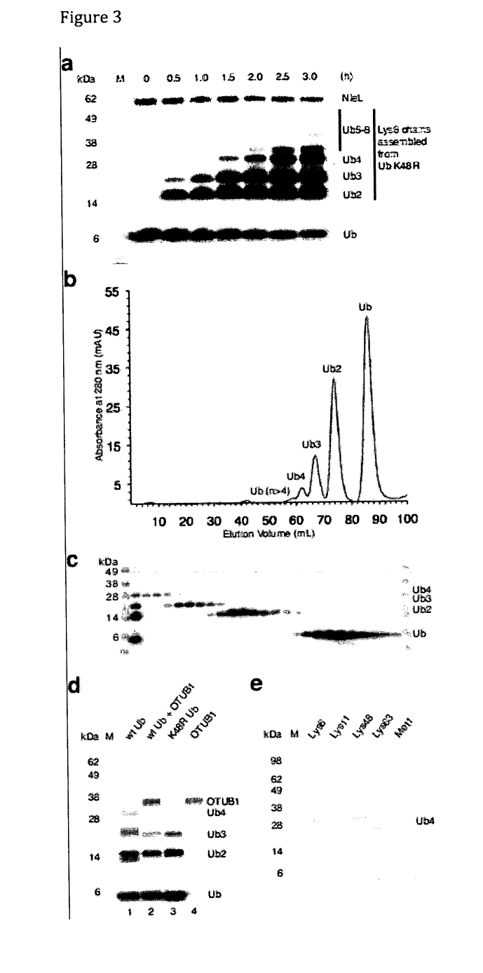

We therefore set out to establish biochemical approaches to assess Ub chain architecture. We reasoned that linkage-specific DUBs could serve as “Ub chain restriction enzymes”, to reveal building blocks comprising distinct linkages (FIG. 2A).

Ubiquitin specific proteases (USPs) hydrolyse all types of chains, and the broad-specificity enzyme USP21cleaved K6- and K48-linked diUb similarly (FIG. 2b); (Ye et al, 2011). In contrast, the ovarian tumour (OTU) family of DUBs comprises enzymes with marked linkage preference. An OTU enzyme with preference for K48-linkages, OTUB1, has been reported (Wang et al, 2009). Importantly, OTUB1 was unable to cleave K6-linked tetraUb under conditions where K48 linked tetraUb was fully hydrolysed (FIG. 2b—shown for diUb). This makes OTUB1 suitable to characterize heterotypic NleL assembled Ub chains.

In contrast, the non-specific viral OTU domain (vOTU) from Crimean Congo Hemorrhagic Fever Virus (CCHFV) (Akutsu, M et al, (2011) P Natl Acad Sci Usa 108, 2228-2233) hydrolysed Lys6- and Lys48-linkages similarly.

However, an enzyme that preferred K6- to K48-linkages has not been reported. We therefore tested a panel of OTU domain DUBs for activity against K6-linked diUb, and discovered that OTUD3, an uncharacterized OTU enzyme, shows strong activity against K6-linkages, but is inefficient against K48-polymers (FIG. 2b).

Together, vOTU, OTUB1 and OTUD3 could be used to analyse Ub chain architecture of NleL assembled Ub chains in vitro. Purified heterotypic penta/hexa Ub (FIG. 2c, lane 1) was cleaved with vOTU, revealing that vOTU can access all linkages within the mixed/branched polymers (FIG. 2c, lanes 13-15). Likewise, OTUB1 in combination with OTUD3 hydrolysed all linkages (FIG. 2c, lanes 10-12). Interestingly, OTUB1 alone disassembled heterotypic Ub chains to sharp bands corresponding to mono, di-, tri-, and tetraUb (FIG. 2c, lanes 4-6). The resulting Ub chains are K6-linked, as they show an identical electrophoretic mobility as K48 Ub chains, and can be cleaved by subsequent treatment with OTUD3.

Strikingly, when the heterogeneous penta/hexaUb input was treated with OTUD3, a different banding pattern was observed, that consisted of mainly of mono- and diUb and a faint signal for triUb (FIG. 2c, lanes 7-9). The remaining K48 chains can be easily distinguished as they show distinct electrophoretic mobility as compared to K6-linked chains (FIG. 2c, compare lane 6 and 8), and are readily cleaved upon incubation with OTUB1.

The distinct hydrolysis profile allows us to conclude that heterotypic penta/hexaUb chains assembled by NleL (i) are heterotypic with most individual polymers comprising more than one linkage type, (ii) are primarily K6-linked and (iii) comprise rarely more than one or two K48 linkage in a row. This suggests that NleL is able to elongate K6-linked Ub chains more efficiently, while it has problems to elongate K48-linkages and stops at diUb. We are unable to distinguish between branched and mixed chains.

We also determined that our technique can sequence branched ubiquitin chains. The pattern observed for OTUD3 cleavage suggested a prevalence of Lys6-linkages in heterotypic chains. Comparison of the assembly of Ub K6R with Ub K48R into homotypic Ub chains over a short time course supported this notion (FIG. 2d). While Lys6-linkages were assembled into long polymers within minutes, assembly of Lys48-linkages was inhibited, stopping at diUb and small amounts of triUb under identical conditions (FIG. 2d). The results from Ub chain sequencing unambiguously revealed heterotypic Ub chains, but could not distinguish whether such Ub chains featured mixed or branched linkages. To test whether NleL can assemble branched species, an assembly reaction was performed with Ub ΔG76 as the acceptor, and Ub K6R/K48R as the donor Ub (FIG. 2e). NleL was capable of assembling branched triUb (FIG. 2d, f), showing that all forms of branched and mixed Ub chains potentially exist in NleL products. Interestingly, Lys6-linkages are also the preferred diUb intermediate on the assembly route to branched triUb species (FIG. 2d, f). The branched triUb species could be purified and analyzed in DUB assays. DUB assays performed as for Ub chain sequencing showed that OTUD3 and OTUB1 hydrolyzed their preferred linkages identically regardless of whether the Ub chain was homotypic or branched (FIG. 2g). Similar results were obtained with DUBs from the USP family (USP7, USP21).

### Example 3

Large Scale Assembly of K6-Linked polyUb

We utilized NleL to perform large-scale enzymatic synthesis of Lys6 polymers for structural analysis. A protocol was devised in which 25 mg of Ub 7 K48R was assembled into unanchored Ub chains of varying lengths that were subsequently separated using size-exclusion chromatography (SEC) (see FIG. 3a-c). This allowed access to homogeneous Lys6 di-, tri- and tetraUb in which each moiety comprises a K48R mutation. To generate wt Lys6 polymers, heterotypic Ub chains were assembled from 25 mg wt Ub, and the reaction was subsequently treated overnight with OTUB1. As established before, OTUB1 was completely inactive against Lys6 polymers, but efficiently removed Lys48 linkages within heterotypic Ub chains. The resulting mixture of Lys6 polymers of different length was purified as previously, and although the yield of longer Ub chains was reduced, milligram quantities of wt Lys6 chains could be produced (FIG. 3d). Lys6 tetraUb can be distinguished from other chain types of this length by its electrophoretic mobility (FIG. 3e).

### REFERENCES

Acconcia F, Sigismund S, Polo S (2009) Ubiquitin in trafficking: the network at work. Exp Cell Res 315: 1610-1618

Akutsu M, Ye Y, Virdee S, Chin J W, Komander D (2011) Molecular basis for ubiquitin and ISG15 cross-reactivity in viral ovarian tumor domains. Proc Natl Acad Sci USA 108: 2228-2233

Al-Hakim A, Escribano-Diaz C, Landry M C, O'Donnell L, Panier S, Szilard R K, Durocher D (2010) The ubiquitous role of ubiquitin in the DNA damage response. DNA Repair 9: 1229-1240

Bang D, Gribenko A V, Tereshko V, Kossiakoff A A, Kent S B, Makhatadze G I (2006) Dissecting the energetics of protein alpha-helix C-cap termination through chemical protein synthesis. Nat Chem Biol 2: 139-143

Ben-Saadon R, Zaaroor D, Ziv T, Ciechanover A (2006) The polycomb protein Ring1 B generates self atypical mixed ubiquitin chains required for its in vitro histone H2A ligase activity. Mol Cell 24: 701-711

Berrow N S, Alderton D, Sainsbury S, Nettleship J, Assenberg R, Rahman N, Stuart D I, Owens R J (2007) A versatile ligation-independent cloning method suitable for high-throughput expression screening applications. Nucleic Acids Res 35: e45

Bremm A, Komander D (2011) Emerging roles for Lys11-linked polyubiquitin in cellular regulation. Trends Biochem Sci 36: 355-363

Chen Z J, Sun L J (2009) Nonproteolytic functions of ubiquitin in cell signaling. Mol Cell 33: 275-286

Dammer E, Peng J (2010) At the crossroads of ubiquitin signaling and mass spectrometry. Expert review of proteomics 7: 643-645

Dammer E B, Na C H, Xu P, Seyfried N T, Duong D M, Cheng D, Gearing M, Rees H, Lah J J, Levey A I, Rush J, Peng J (2011) Polyubiquitin linkage profiles in three models of proteolytic stress suggest the etiology of Alzheimer disease. J Biol Chem 286: 10457-10465

Datta A B, Hura G L, Wolberger C (2009) The structure and conformation of Lys63-linked tetraubiquitin. J Mol Biol 392: 1117-1124

Deshaies R J, Joazeiro C A (2009) RING domain E3 ubiquitin ligases. Annu Rev Biochem 78: 399-434

Dikic I, Wakatsuki S, Walters K J (2009) Ubiquitin-binding domains—from structures to functions. Nat Rev Mol Cell Biol 10: 659-671

Dye B T, Schulman B A (2007) Structural mechanisms underlying posttranslational modification by ubiquitin-like proteins. Annu Rev Biophys Biomol Struct 36: 131-150

Edelmann M J, Iphofer A, Akutsu M, Altun M, di Gleria K, Kramer H B, Fiebiger E, Dhe-Paganon S, Kessler B M (2009) Structural basis and specificity of human otubain 1-mediated deubiquitination. Biochem J 418: 379-390

Hershko A, Ciechanover A (1998) The ubiquitin system. Annu Rev Biochem 67: 425-479

Iwai K, Tokunaga F (2009) Linear polyubiquitination: a new regulator of NF-kappaB activation. EMBO Rep 10: 706-713

Komander D (2009) The emerging complexity of protein ubiquitination. Biochem Soc Trans 37: 937-953

Komander D, Clague M J, Urbé S (2009a) Breaking the chains: structure and function of the deubiquitinases. Nat Rev Mol Cell Biol 10: 550-563

Komander D, Reyes-Turcu F, Licchesi J D, Odenwaelder P, Wilkinson K D, Barford D (2009b) Molecular discrimination of structurally equivalent Lys 63-linked and linear polyubiquitin chains. EMBO Rep 10: 466-473

Lange O F, Lakomek N-A, Farés C, Schröder G F, Walter K F A, Becker S, Meiler J, Grubmüller H, Griesinger C, de Groot B L (2008) Recognition dynamics up to microseconds revealed from an RDC-derived ubiquitin ensemble in solution. Science 320: 1471-1475

Lin D Y, Diao J, Zhou D, Chen J (2010) Biochemical and structural studies of a HECT-like ubiquitin ligase from E. coli O157:H7. J Biol Chem

Morris J R, Solomon E (2004) BRCA1:BARD1 induces the formation of conjugated ubiquitin structures, dependent on K6 of ubiquitin, in cells during DNA replication and repair. Hum Mol Genet 13: 807-817

Nishikawa H, Ooka S, Sato K, Arima K, Okamoto J, Klevit R E, Fukuda M, Ohta T (2004) Mass spectrometric and mutational analyses reveal Lys-6-linked polyubiquitin chains catalyzed by BRCA1-BARD1 ubiquitin ligase. J Biol Chem 279: 3916-3924

Pickart C M, Raasi S (2005) Controlled synthesis of polyubiquitin chains. Methods Enzymol 399: 21-36

Piscatelli H, Kotkar S A, McBee M E, Muthupalani S, Schauer D B, Mandrell R E, Leong J M, Zhou D (2011) The EHEC type III effector NleL is an E3 ubiquitin ligase that modulates pedestal formation. PloS one 6: e19331

Randow F, Lehner P J (2009) Viral avoidance and exploitation of the ubiquitin system. Nat Cell Biol 11: 527-534

Reyes-Turcu F E, Ventii K H, Wilkinson K D (2009) Regulation and cellular roles of ubiquitin-specific deubiquitinating enzymes. Annu Rev Biochem 78: 363-397

Rotin D, Kumar S (2009) Physiological functions of the HECT family of ubiquitin ligases. Nat Rev Mol Cell Biol 10: 398-409

Virdee S, Ye Y, Nguyen D P, Komander D, Chin J W (2010) Engineered diubiquitin synthesis reveals Lys29-isopeptide specificity of an OTU deubiquitinase. Nat Chem Biol 6: 750-757

Wang T, Yin L, Cooper E M, Lai M Y, Dickey S, Pickart C M, Fushman D, Wilkinson K D, Cohen R E, Wolberger C (2009) Evidence for bidentate substrate binding as the basis for the K48 linkage specificity of otubain 1. J Mol Biol 386: 1011-1023

Wenzel D M, Lissounov A, Brzovic P S, Klevit R E (2011) UBCH7 reactivity profile reveals parkin and HHARI to be RING/HECT hybrids. Nature 474: 105-108

Wu-Baer F, Lagrazon K, Yuan W, Baer R (2003) The BRCA1/BARD1 heterodimer assembles polyubiquitin chains through an unconventional linkage involving lysine residue K6 of ubiquitin. J Biol Chem 278: 34743-34746

Xu P, Duong D M, Seyfried N T, Cheng D, Xie Y, Robert J, Rush J, Hochstrasser M, Finley D, Peng J (2009) Quantitative proteomics reveals the function of unconventional ubiquitin chains in proteasomal degradation. Cell 137: 133-145

Ye Y, Akutsu M, Reyes-Turcu F, Enchev R I, Wilkinson K D, Komander D (2011) Polyubiquitin binding and cross-reactivity in the USP domain deubiquitinase USP21. EMBO Rep 12: 350-357

Unless otherwise stated, all technical and scientific terms used herein have the same meanings as commonly understood by one of ordinary skill in the art to which this invention belongs. Any methods and materials similar or equivalent to those described herein can be used in the practice or testing of the present invention. Methods, devices, and materials suitable for such uses are described above. All publications cited herein are incorporated herein by reference in their entirety for the purpose of describing and disclosing the methodologies, reagents, and tools reported in the publications that might be used in connection with the invention.

1. A method for producing free polyubiquitin chains linked through a single desired lysine residue, comprising the steps of: (a) selecting an E3 ubiquitin ligase enzyme which is homologous to mammalian HECT E3 ligases and possesses the desired lysine residue specificity; (b) incubating the E3 enzyme with an E1 ubiquitin activating enzyme, an E2 ubiquitin conjugating enzyme and monomeric ubiquitin; and (c) if undesired linkages are present, removing the undesired linkages by exposure to a deubiquitinase (DUB) enzyme having the appropriate specificity.

2. The method according to claim 1, wherein the monomeric ubiquitin is modified to prevent undesired linkage formation.

3. The method according to claim 1, wherein the E3 ubiquitin ligase enzyme is NleL.

4. The method according to claim 3, wherein the single desired linkage is K6.

5. The method according to claim 4, wherein the DUB enzyme is OTUB1.

6. The method according to claim 1, wherein the single desired linkage is K48.

7. The method according to claim 1, wherein the E2 enzyme is Ube2L3/UbcH7.

8. The method according to claim 2, wherein the monomeric ubiquitin is a K48R mutant.

9. A kit for preparing polyubiquitin chains having a desired linkage specificity, comprising an E3 ubiquitin ligase enzyme which is homologous to mammalian HECT E3 ligases, an E1 ubiquitin activating enzyme, an E2 ubiquitin conjugating enzyme and a deubiquitinase (DUB) enzyme.

10. The kit according to claim 9, wherein the E3 Ubiquitin ligase enzyme is NleL.

11. The kit according to claim 10, wherein the single desired linkage is K6.

12. The kit according claim 9, wherein the DUB enzyme is OTUB1.

13. The kit according to claim 9, wherein the single desired linkage is K48.

14. The kit according claim 9, wherein the E2 enzyme is Ube2L3/UbcH7.

15. The method according to claim 2, wherein the single desired linkage is K48.

16. The method according to claim 3, wherein the single desired linkage is K48.

17. The method according to claim 2, wherein the E2 enzyme is Ube2L3/UbcH7.

18. The method according to claim 3, wherein the E2 enzyme is Ube2L3/UbcH7.

19. The method according to claim 4, wherein the E2 enzyme is Ube2L3/UbcH7.

20. The method according to claim 5, wherein the E2 enzyme is Ube2L3/UbcH7.

21. The method according to claim 6, wherein the E2 enzyme is Ube2L3/UbcH7.

22. The kit according to claim 10, wherein the DUB enzyme is OTUB1.

23. The kit according to claim 11, wherein the DUB enzyme is OTUB1.

24. The kit according to claim 10, wherein the single desired linkage is K48.

25. The kit according to claim 10, wherein the E2 enzyme is Ube2L3/UbcH7.

26. The kit according to claim 11, wherein the E2 enzyme is Ube2L3/UbcH7.

27. The kit according to claim 12, wherein the E2 enzyme is Ube2L3/UbcH7.

28. The kit according to claim 13, wherein the E2 enzyme is Ube2L3/UbcH7.
